(19) United States
(12) Patent Application Publication
(10) Pub. No.: US 2002/0029082 A1
(43) Pub. Date: Mar. 7, 2002

(54) Vertebral spacer and method of use

(76) Inventor: Nabil L. Muhanna, Gainesville, GA (US)

Correspondence Address: WOMBLE CARLYLE SANDRIDGE & RICE, P.O. Box 7037, Atlanta, GA, 30357-0037, US

(21) Appl. No.: 09/941,040

(22) Filed: Aug. 28, 2001

Related U.S. Application Data
(60) Provisional application No. 60/228,694, filed on Aug. 29, 2000.

(51) Int. Cl. A61F 2/44

(52) U.S. Cl. 623/17.11; 623/17.16

(57) ABSTRACT

The present invention provides a novel vertebral spacer for supporting adjacent vertebrae. The vertebral spacer has a body having an anterior face and a posterior face extending between an upper surface and a lower surface. The heights of the anterior face and the posterior face may differ, the non-parallel upper and lower surfaces thereby maintaining the curvature of the spine when the vertebral spacer is inserted between two vertebrae. A stabilizing body may be connected to the body of the spacer by an attachment member that optionally allows the body and the stabilizing body to rotate relative to each other. The vertebral spacer may have a locking assembly whereby the body and the stabilizing body may be rigidly locked. The vertebral spacer may include optional channels extending through the body of the spacer that facilitate tissue ingrowth and bony fusion between the adjacent vertebrae. A stabilizing body connected to the body of the vertebral spacer may be formed by delivering a biocompatible liquid polymer material into a liquid receiving bore and a transverse bore. Excess polymer may seep into the intervertebral space where it hardens to form the stabilizing body extending from the body of the spacer. A method of inserting the vertebral spacer into an intervertebral space is also described. The body of the vertebral spacer is rotated so that the lower surface and the upper surface of the body contact the adjacent vertebrae, whereupon the body is secured by attaching (if necessary), and locking, the stabilizing body.

The present application claims the benefit of the provisional U.S. Application Ser. No. 60/228,694 filed Aug. 29, 2000, which is incorporated herein by reference.

The present invention generally relates to a vertebral spacer to be inserted into an intervertebral space, thereby supporting the spinal column of a patient. The present invention further relates to methods for implanting the vertebral spacer into the spinal column and securing the spacer therein.

### BACKGROUND OF THE INVENTION

The spinal column, which is the central support to the vertebrate skeleton and a protective enclosure for the spinal cord, is a linear series of bones, or vertebrae. Intervertebral discs separate and reduce friction between adjacent vertebrae and absorb compression forces applied to the spinal column. Spinal nerves that extend from each side of the spinal cord exit the column at intervertebral forama.

A typical vertebra comprises an anterior body, and a posterior arch that surrounds the spinal cord lying within the vertebral foramen formed by the arch. The muscles that flex the spine are attached to three processes extending from the posterior arch. On the upper surface of each vertebra in a standing human, are two superior articulated processes that oppose two inferior articulated processes extending from the lower surface of an adjacent vertebra. Facets on the opposing processes determine the range and direction of movement between adjacent vertebrae, and hence the flexibility of the spinal column.

The intervertebral discs include the fibrillar cartilage of the anulus fibrosus, a fibrous ring, the center of which is filled with an elastic fibrogelatinous pulp that acts as a shock absorber. The outer third of the anulus fibrosus is innervated. The entire spinal column is united and strengthened by encapsulating ligaments.

Back pain is one of the most significant problems facing the workforce in the United States today. It is a leading cause of sickness-related absenteeism and is the main cause of disability for people aged between 19 and 45. Published reports suggest that the economic cost is significant, treatment alone exceeding $80 billion annually. Although acute back pain is common and typically treated with analgesics, chronic pain may demand surgery for effective treatment.

Back pain can occur from pinching or irritation of spinal nerves, compression of the spine, vertebral shifting relative to the spinal cord axis, and bone spur formation. The most common cause of disabling back pain, however, stems from trauma to a intervertebral disc, resulting from mechanical shock, stress, tumors or degenerative disease, which may impair functioning of the disc and limit spinal mobility. In many cases, the disc is permanently damaged and the preferred treatment becomes partial or total excision.

Another cause of back injury is herniation of the intervertebral disc, wherein the gelatinous fluid of the nucleus pulposus enters the vertebral canal and pressures the spinal cord. Again, surgery is often the only method available for permanent relief from pain or the neurological damage ensuing from the pressure of fluid on the spinal cord, and requires replacement of the damaged disc.

Traumatic injury to an intervertebral disc that is not removed will frequently promote scar tissue formation. Scar tissue is weaker than original healthy tissue so that the disc will progressively degenerate, lose water content, stiffen and become less effective as a shock absorber. Eventually, the disc may deform, herniate, or collapse, limiting flexibility of the spinal column at that position. The only option is for the intervertebral disc to be partially or totally removed.

When the disc is partially or completely removed, it is necessary to replace the excised material to prevent direct contact between hard bony surfaces of adjacent vertebrae. One vertebral spacer that may be inserted between adjacent vertebrae, according to U.S. Pat. No. 5,989,291 to Ralph et al., includes two opposing plates separated by a belleville washer or a modified belleville washer. The washer functions to provide a restorative force to mimic the natural functions of the disc of providing a shock absorber and mobility between adjacent vertebrae. However, mechanical devices intended to replicate intervertebral disc function have had only limited success. An alternative approach is a “cage” that maintains the space usually occupied by the disc to prevent the vertebrae from collapsing and impinging the nerve roots.

Spinal fusion may be used to restrict the motion, between two vertebrae, that comes from segmental instability. Fusing the vertebrae together, however, reduces the mechanical back pain by preventing the now immobile vertebrae from impinging on the spinal nerve. The disadvantage of such spacers is that stability is created at the expense of the flexibility of the spine.

Surgical procedures for replacing intervertebral disc material, rather than fusing of the vertebrae, have included both anterior approaches and posterior approaches to the spinal column. The posterior approach (from the back of the patient) encounters the spinous process, superior articular process, and the inferior articular process that must be removed to allow insertion of the disc replacement material into the intervertebral space. The excess removal of the bony process triggers further degradation and impediment of the normal movement of the spine. The anterior approach to the spinal column is complicated by the internal organs that must be bypassed or circumvented to access the vertebrae.

Many intervertebral spacers require preparation of the surfaces of the adjacent vertebrae to accommodate the spacer, causing significant tissue and bone trauma. For example, chiseling or drilling of the vertebral surface may be required to prepare a receiving slot. They may also require screwing the spacer into the intervertebral space, making installation difficult and increasing trauma to the vertebral tissue. Many spacers include complex geometries and are costly to manufacture. Examples of such geometrically complex spacers are described in U.S. Pat. No. 5,609,636 to Kohrs et al., U.S. Pat. No. 5,780,919 to Zdeblick et al., U.S. Pat. No. 5,865,848 to Baker and U.S. Pat. No. 5,776,196 to Matsuzaki et al. Many of these complex spacers may require screwing the spacer into the intervertebral space, thereby making installation difficult and traumatic to the vertebral tissue.

### SUMMARY OF THE INVENTION

There is a need for a vertebral spacer having a simple geometry that is easily insertable into an intervertebral space while causing minimal trauma to the surface of the vertebrae as well as the bony processes thereof. The present invention provides a vertebral spacer having a simple geometry for supporting adjacent vertebrae after excision, at least partially or wholly, of an intervertebral disc. The spacer includes a body having a lower surface and an upper surface. The lower surface will be supported by a lower vertebra; the upper surface will support the adjacent upper vertebra. The body of the vertebral spacer of the present invention, therefore, provides support between the two adjacent vertebrae and to the spinal column.

The body of the vertebral spacer of the present invention additionally has an anterior face and a posterior face extending from the lower surface. The height of the anterior face of the body may be greater than the height of the posterior face to maintain the curvature of the spine when the vertebral spacer is inserted between two vertebrae. A stabilizing body is connected to and extends from the body of the spacer. The stabilizing body may be connected to the body of the spacer by an attachment member that optionally allows the body and the stabilizing body to rotate relative to each other. The present invention further provides a vertebral spacer having a locking assembly whereby the body and the stabilizing body, once orientated to a desired position with a spinal column, and relative to each other, may be rigidly locked.

The present invention further contemplates the optional use of one or more channels extending through the body of the spacer to facilitate tissue ingrowth and bony fusion between the adjacent vertebrae.

In one embodiment of the present invention, the stabilizing body may be formed by delivering a biocompatible liquid polymer material into a liquid receiving bore and a transverse bore. The polymer material is injected in an amount sufficient to fill the bore and to pass out of at least one transverse bore that communicates with the liquid receiving bore. The excess polymer seeps into the space between adjacent vertebrae. Hardening of the liquid polymer material then forms the stabilizing body extending from the body of the spacer.

The present invention further provides a method of maintaining a separation distance between adjacent vertebrae. At least one vertebral spacer according to the present invention can be inserted into an intervertebral space to support the adjacent vertebrae. The body of the vertebral spacer may be inserted into the receiving intervertebral space in an orientation that reduces contact between the spacer and the adjacent vertebrae. Once inserted into the selected position, the body of the vertebral spacer may be rotated so that the lower surface and the upper surface of the body contact the adjacent vertebrae, whereupon the body is secured by attaching (if necessary) and locking the stabilizing body.

Various objects, features, and advantages of the invention will become more apparent upon review of the detailed description set forth below when taken in conjunction with the accompanying drawing figures, which are briefly described as follows.

### BRIEF DESCRIPTION OF THE FIGURES

FIG. 1 is a perspective view of a vertebral spacer according to one embodiment of the present invention.

FIG. 2 is an exploded perspective view of the vertebral spacer of FIG. 1 showing the spacer in an unassembled manner.

FIG. 3 is an end view of the embodiment according to FIG. 1.

FIG. 4 is a perspective view of another embodiment of the vertebral spacer according to the present invention, illustrating a body rotatably connected to a stabilizing body.

FIG. 5 is a cross-sectional side view of the vertebral spacer taken along lines 5-5 of FIG. 4.

FIG. 6 is a cross-sectional side view of an embodiment of the locking assembly comprising a locking pin.

FIG. 7 is an exploded perspective view of the vertebral spacer of FIG. 4 showing the vertebral spacer in an unassembled manner.

FIG. 8 is a perspective view of the vertebral spacer according to FIG. 4 showing a preinsertion orientation of the vertebral spacer body relative to the stabilizing body.

FIG. 9 is a perspective view according to another embodiment of the vertebral spacer of the present invention, showing triangular protrusions on the vertebral spacer body.

FIG. 10 is a side view of the embodiment of the vertebral spacer having triangular protrusions on the vertebral spacer body.

FIG. 11 is a perspective view of another embodiment of the vertebral spacer of the present invention, showing rounded protrusions on the vertebral spacer body.

FIG. 12 is a side view of the embodiment of the vertebral spacer having rounded protrusions on the vertebral spacer body.

FIG. 13 is a perspective view of an embodiment of the vertebral spacer according to the present invention having an ellipsoidal body.

FIG. 14 is an end view of the vertebral spacer having an ellipsoidal body.

FIG. 15 is an exploded perspective view of the vertebral spacer having an ellipsoidal body, showing the vertebral spacer in an unassembled manner.

FIG. 16 is a perspective view of an embodiment of the vertebral spacer according to the present invention having channels and a liquid receiving bore and communicating traverse bore system for receiving a liquid polymer material.

FIG. 17 is a perspective view of the vertebral spacer according to FIG. 16 showing a stabilizing body formed by a liquid polymer material extruded from a traverse bore and hardening in situ.

FIG. 18 is a cross-sectional view of the vertebral spacer according FIG. 16.

FIG. 19 is an exploded view of an embodiment of the vertebral spacer of the present invention having a leaf spring locking assembly.

FIG. 20 is a perspective view of the assembled vertebral spacer having a leaf spring locking assembly.

FIG. 21 is a side-elevation of the lower region of the human spinal column, showing the body of the vertebral spacer according to the present invention inserted between adjacent vertebrae and before rotation to substantially contact adjacent vertebrae.

FIG. 22 is a side-elevation of the lower region of the human spinal column, showing the body of the vertebral spacer according to the present invention rotated to contact and support adjacent vertebrae.

### DETAILED DESCRIPTION OF THE INVENTION

A full and enabling disclosure of the present invention, including the best mode known to the inventor of carrying out the invention, is set forth more particularly in the remainder of the specification, including reference to the accompanying drawings, wherein like reference numerals designate corresponding parts throughout several figures. This description is made for the purpose of illustrating the general principles of the invention and should not be taken in the limiting sense.

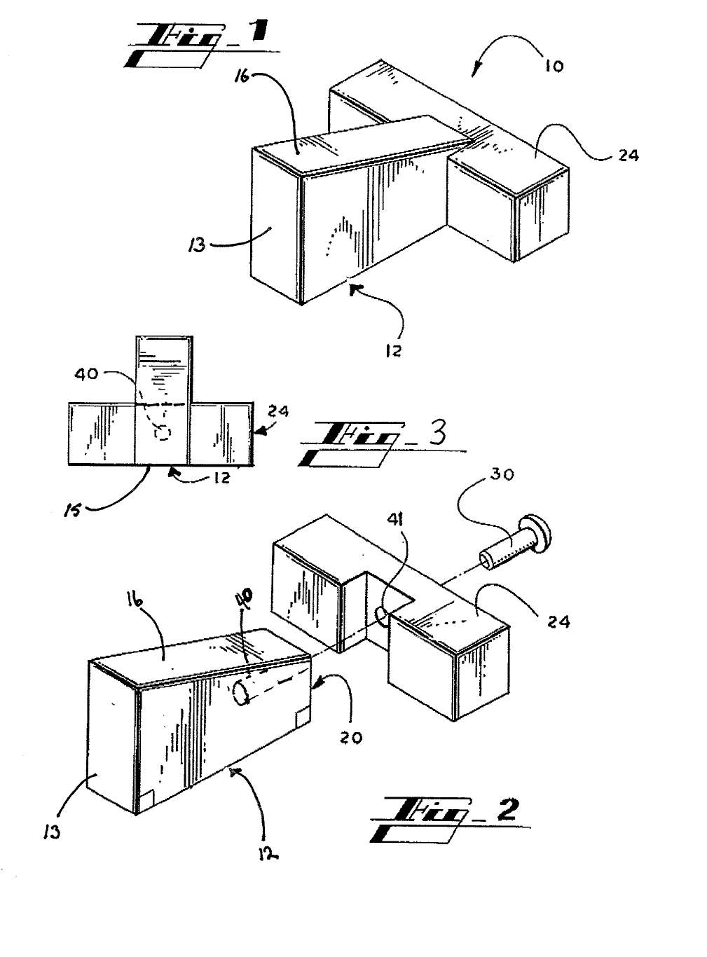

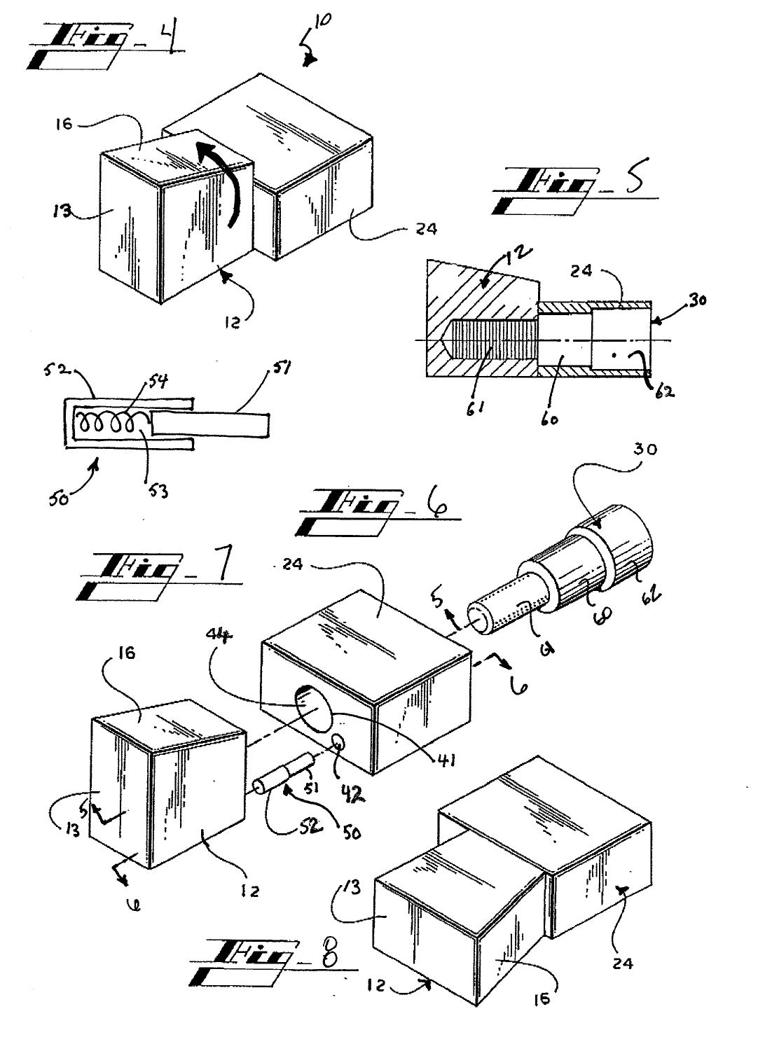

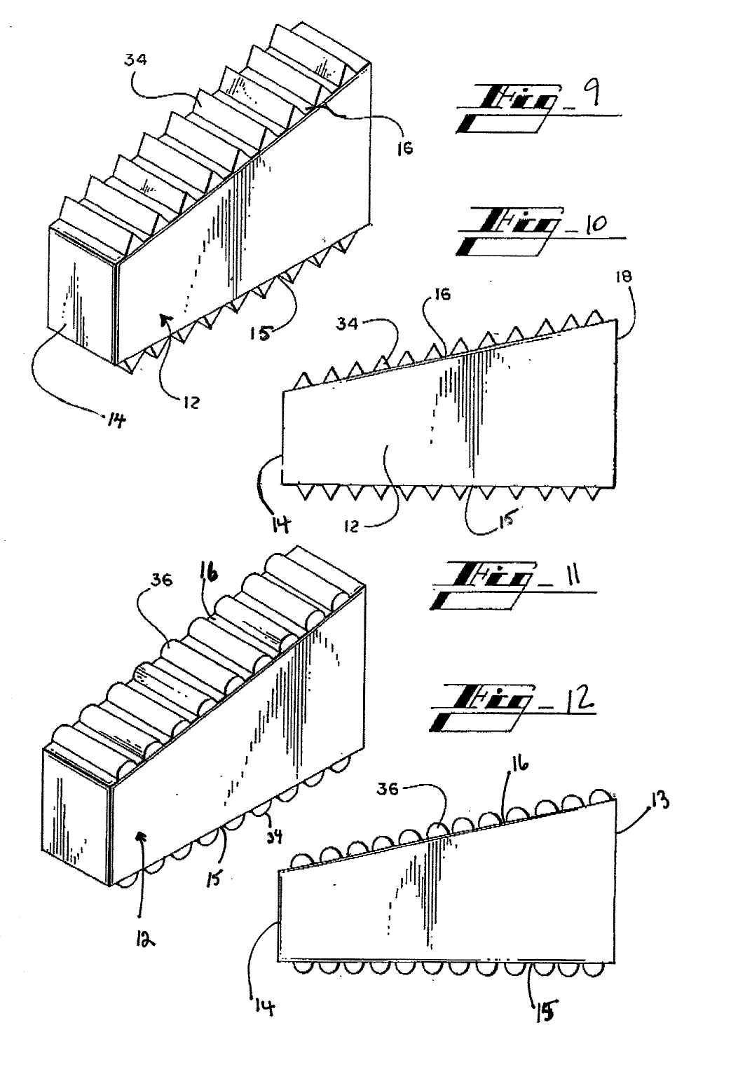

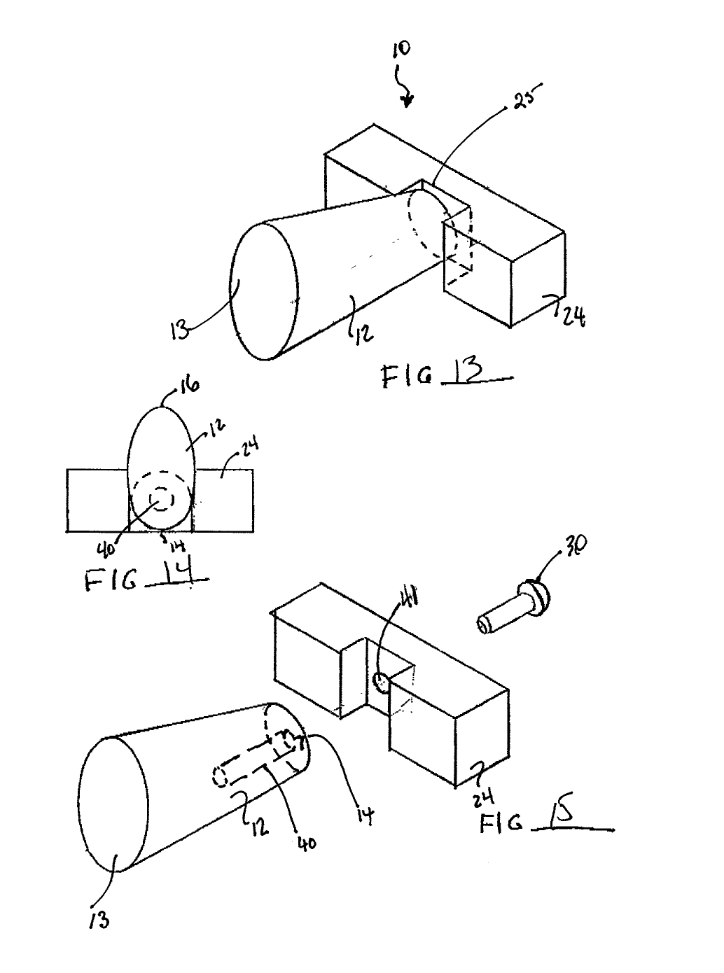

Examples of the vertebral spacer 10 in accordance with the present invention are shown in FIGS. 1-20. As shown in FIG. 22, the vertebral spacers 10 of the present invention support adjacent vertebrae 20 after partial or total surgical excision of an intervertebral disc 21, thereby preventing collapse and/or compression in this region of the spine that might otherwise lead to sever neurological damage. The vertebral spacer 10 of the present invention is useful to replace an intervertebral disc 21 that has degenerated due to traumatic injury, vertebral displacement, disease such as, for example, autoimmune disease or rheumatoid arthritis or any other pathological condition of the spinal column that may injure or shift the intervertebral disc. The vertebral spacers 10 of the present invention provide support to the vertebrae 20 and maintain the distance between vertebrae and preserve the natural curvature of the spine.

The vertebral spacer 10 of the present invention includes a body 12 having a first bore 40, as shown in FIGS. 2 and 3. The body 12 is adapted to fit within an intervertebral space 23 between adjacent vertebrae 20. A stabilizing body 24 having a second bore 41 with an interior surface 44 extends from the body 12 and is adapted to retain the body 12 within the intervertebral space 23. The body 12 may be attached to the stabilizing body 24 by an attachment member 30. The stabilizing body 24 may be rotatably attached to the body 12 by slideably disposing the attachment member 30 through the second bore 41 of the stabilizing body 24. It is contemplated that the stabilizing body 24 may be optionally attached to the body 12, and connected thereto by the attachment member 30 before inserting the vertebral spacer 10 into a patient. Alternatively, the stabilizing body 24 may be attached after the body 12 has been inserted into the patient.

The attachment member 30 may be any device that will connect the body 12 to the stabilizing body 24. Suitable devices particularly useful in the present invention, however, include a pin, a bolt, a threaded pin or bolt and the like. One example of an attachment member 30 is shown in FIG. 2 comprising a shaft 26 and a head 27. In another example, shown in FIG. 6, the attachment member 30 comprises an anchoring region 28, a rotating region 29, and a head 27. It is contemplated, however, that the anchoring region 28 may be threaded for engaging a like thread in the first bore 40 for securing the anchoring region 28 therein. It is also contemplated that any means known to one of skill in the art may be employed to secure the anchoring region 28 to the body 12 including, but not limited to, interlocking screw threads, an adhesive, a leaf spring lock or any other method that will rigidly connect the body 12 to the attachment member 30.

Once the body 12 has been rotated into the desired position relative to the adjacent vertebrae 20, the stabilizing body may be attached to the body 12, or if already attached thereto, the vertebral spacer 10 may be locked to form a rigid assembly. In one embodiment of the vertebral spacer 10 of the present invention, as shown in FIGS. 1-3, the body 12 may enter a recess 25 of the stabilizing body 24, the recess resisting further rotation of the body 12 relative to the stabilizing body 24.

It is contemplated that the vertebral spacer 10 may be of any biocompatible or physiologically inert material or combination of such materials having the mechanical strength capable of maintaining the intervertebral space 23 between two adjacent vertebrae 20 when inserted therein. The material of the body 12 and stabilizing body 24 of the vertebral spacer 10 of the present invention may or may not be identical and may be rigid such as a metal, a rigid plastic or the like. Examples of such materials include bone, titanium, titanium alloy, stainless steel, chrome cobalt, and polymeric materials such as methyl methacrylate (MMA), urethane, polyacetal and the like. The material of the vertebral spacer 10 may, however, also have a degree of resilience and thereby tolerate a degree of compression. Such materials may include, but are not limited to, polymers such as carbon fiber reinforced polymer such as PEEK (polyetherether ketone), polycarbonate, polypropylene, polyethylene, polyamide and silicone-based polymers.

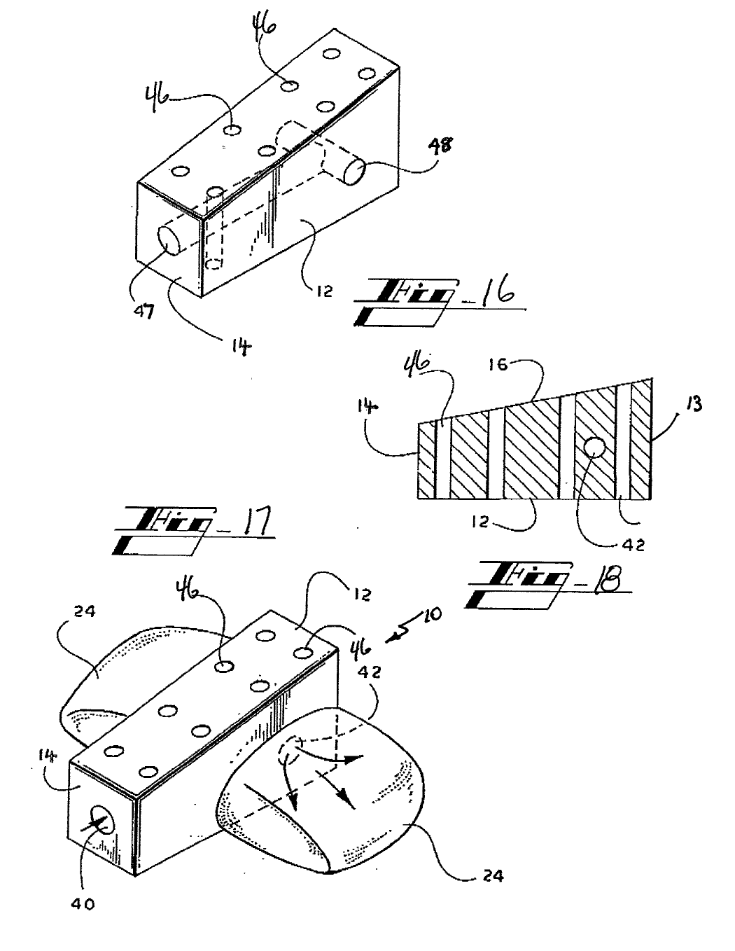

The body 12 of the vertebral spacer 10 of the present invention may have any conformation that will allow the body 12 to be positioned in an intervertebral space 23 between adjacent vertebrae 20 and will subsequently maintain an intervertebral space when in a desired position. Suitable geometric cross-sections that may be applied to the body 12 include, for example, a rectangular cross-section, a trapezoidal cross-section, a circular cross-section, an elliptical cross-section or the like. In one embodiment of the vertebral space 10 of the present invention, the body 12 has a rectangular transverse cross-section as shown, for example, in FIGS. 1-3. In another embodiment of the vertebrate spacer of the present invention alternative configuration, the body 12 may have an anterior face 13 and a posterior face 14 and have a circular or an ellipsoidal cross-section, as shown in FIGS. 13-15, or a combination thereof.

The vertebral spacer 10 of the present invention may have a plurality of surfaces, including a lower surface 15 and an upper surface 16, the lower surface 15 having an anterior face 13 and a posterior face 14 extending therefrom, as shown in FIG. 1. The anterior face 13 may be directed towards the inner body cavity of a patient, and the posterior face 14 may be directed towards the dorsal surface of the patient. The vertebral spacer 10 can be configured such that the height of the anterior face 13 is greater than the height of the posterior face 14, as is illustrated, for example, in FIG. 1. The difference in the height of the opposing anterior 13 and posterior 14 faces of the vertebral spacer 10 of the present invention, so that the lower surface 15 and the upper surface 16 are non-parallel, is useful to preserve the natural curvature of the spinal column.

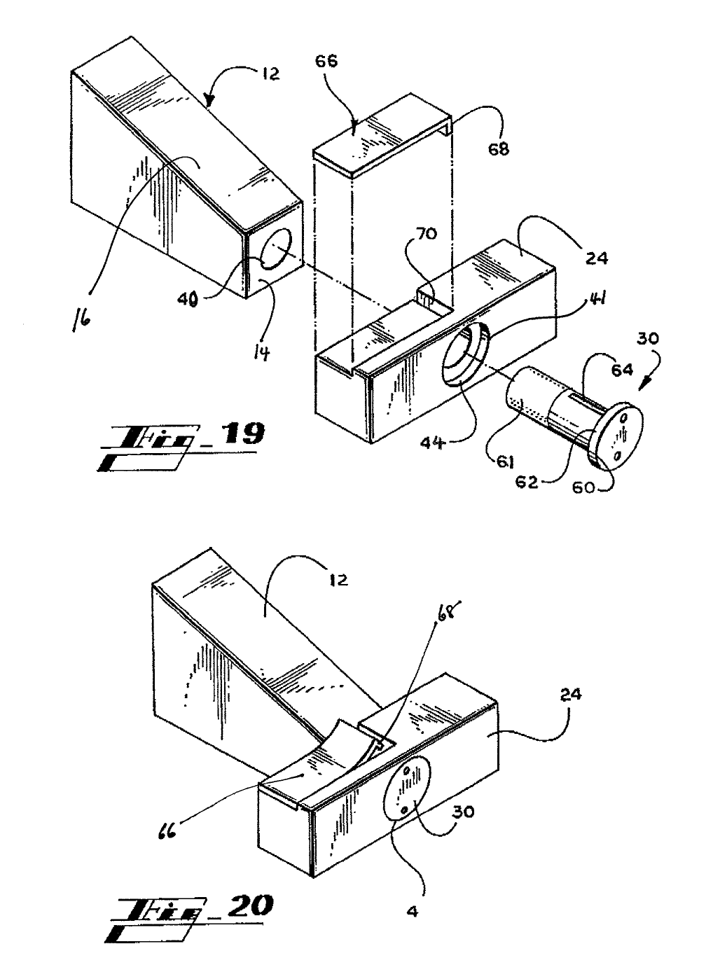

The body 12 of the vertebral spacer 10 of the present invention may further include a channel 46 or a plurality of channels 46 extending through the body 12 such as, for example, shown in FIGS. 16-18. Bony or other tissue growth from adjacent vertebrae 20 that extends into the channels 46 of the vertebral spacer 10 of the present invention may unite and effectively fuse the adjacent vertebrae 28. It is further contemplated that a tissue growth factor or an osteogenic material may be inserted into the apertures to facilitate this fusion. Suitable growth factors include, but are not limited to, growth hormones, steroids, tissue growth factors and the like. Alternatively, the channel 46 or plurality of channels 46 may only partially extend into the body 12. While not fusing the adjacent vertebrae 20, therefore, the penetrating tissue growth will stabilize the vertebral spacer 10 within the intervertebral space 23.

The lower surface 15 and the upper surface 16 of the vertebral spacer 10 optionally includes at least one protrusion 34 on the lower surface 15 and/or on the upper surface 16 for frictionally engaging the adjacent vertebrae 20. Exemplary embodiments of the protrusions 34 of the present invention are illustrated in FIGS. 9-12. It is contemplated that the body 12 may have a single protrusion 34, or a plurality of protrusions 34 as shown in FIGS. 9-12. The protrusions 34 may have any suitable geometric configuration that will allow the body 12 of the vertebral spacer 10 of the present invention to be secured to adjacent vertebrae 20, including having a triangular, rounded, or rectangular cross-section and the like, or any combination thereof. The protrusions may be elongated as shown in FIGS. 9-12, or any other shape such as square or circular protrusions or irregular protrusions not elongated.

As shown in FIGS. 6-8, 19 and 20, embodiments of the vertebral spacer 10 of the present invention may further include a locking assembly whereby, once the vertebral spacer 10 has been inserted into an intervertebral space and the body 12 has been rotated into a desired position, the body 12 is locked relative to the stabilizing body 24. In one exemplary embodiment of the vertebral spacer 10 of the present invention, the locking assembly is a locking pin 50 having a male member 51, a female member 52, the female member 52 having a lumen 53, and a spring 54 therein. The male member 51 is slideably disposed within the lumen 53 of the female member 52. When the male member 51 is pushed into the lumen 53, it encounters a resistant force exerted by compression of the spring 54.

In the embodiment of the vertebral spacer 10 of the present invention, as shown in FIG. 6, the body 12 further includes a first pin receiving bore 42 capable of accepting the female member 52 of the locking pin 50. The stabilizing body 24 has a second pin receiving bore 43 capable of receiving the male member 51 of the locking pin 50.

Rotation of the body 12 relative to the stabilizing body 24 by rotation of the rotating region 29 of the attachment member 30 within the second bore 41, aligns the first pin receiving bore 42 and the second pin receiving bore 43. The compressed spring 54 will expand and push the male member 51 of the locking pin 50 partially into the second pin receiving bore 43, thereby preventing further rotation of the body 12 relative to the stabilizing body 24. In an alternative exemplary embodiment of the present invention, the male member 51 of the locking pin 50 is within the second pin receiving bore 43. The first pin receiving bore 42 receives the female member 52 of the locking pin 50.

In yet another embodiment of the vertebral spacer 10 of the present invention, the first pin receiving bore 42 of the body 12 has a spring 54 therein and a locking pin 50 that upon alignment of the first and the second pin receiving bores, 42 and 43 respectively, will push the locking pin 50 into the first pin receiving bore 42. A portion of the locking pin 50 is retained within the second pin receiving bore 43, thereby locking movement of the body 12 relative to the stabilizing body 24.

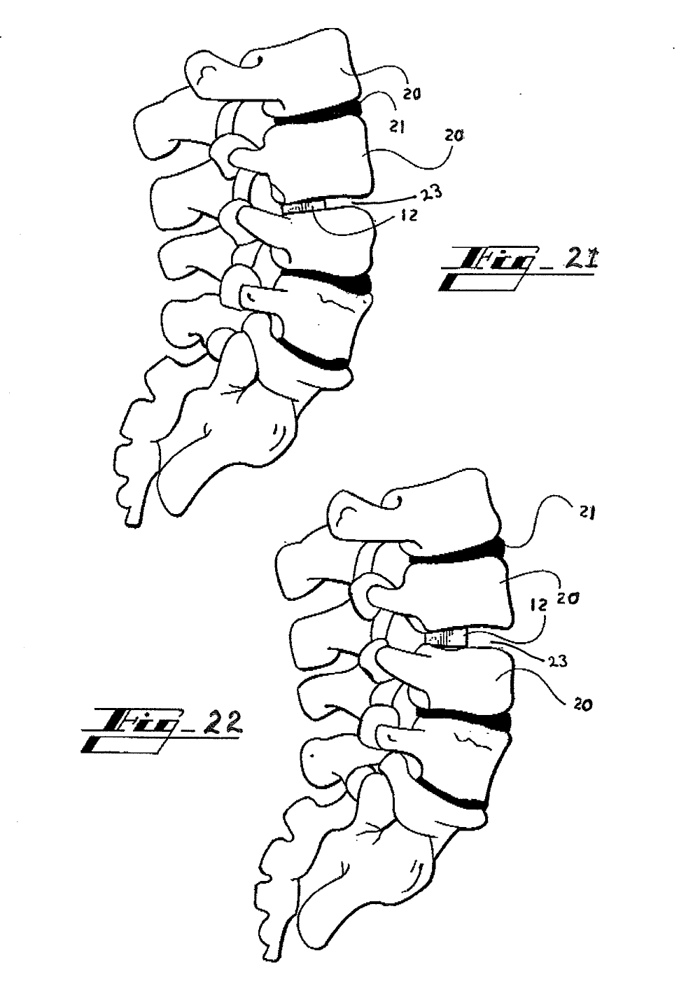

In still another embodiment of the vertebral spacer 10 of the present invention, as schematically illustrated in FIGS. 19 and 20, the stabilizing body 24 of this embodiment further includes a locking assembly having a communicating slot 70 connecting surface 17 of the stabilizing body 24 and the interior surface 44 of the second bore 41. Attached to the surface 17 is a leaf spring 66 having a locking arm 68 capable of slideably entering through the communicating slot 70, and thereby extending into the interior of the second bore 41. This embodiment of the present invention further comprises the attachment member 30 having an anchoring region 28, the rotating region 29 and a head 27. The rotating region 29 has a receiving notch 64 configured to receive the locking arm 68 of the leaf spring 66.

The attachment member 30 is initially positioned so that the locking arm 68, which is slideably disposed in the communicating slot 70, and the receiving notch 64 are not aligned. The body 12 and the attachment member 30 secured thereto may be rotated relative to the stabilizing body 24 to place the body 12 in a desired position within the intervertebral space 23. The rotation will align the locking arm 68 and the receiving notch 64 whereupon the leaf spring 66 will depress the locking arm 68 into the receiving notch 64, and locking the body 12 and the stabilizing body 24 into the selected configuration.

In another embodiment of the vertebral spacer 10 of the present invention, the stabilizing body 24 extending from the body 12 is a hardened biocompatible liquid delivered to the body 12 of the vertebral spacer 10 once the vertebral spacer 10 has been inserted in the spinal column of the patient. Referring now to FIGS. 16-18, in this embodiment, the body 12 has a liquid receiving bore 47 that extends from the posterior face 14 of the body 12 to an intermediate position within the body 12. It is further contemplated, however, that the liquid receiving bore 47 may extend from any surface of the body 12 that will allow delivery of a liquid thereto. The body 12 further includes at least one transverse bore 48 communicating with the liquid receiving bore 47 and with an exterior surface of the body 12.

In this embodiment, once the vertebral spacer 10 is inserted between the adjacent vertebrae 20, a biocompatible liquid polymer material is delivered into the liquid receiving bore 47 in an amount greater than the volume of the liquid receiving bore 47. Excess liquid polymer material flows from the liquid receiving bore 47 into the communicating transverse bore 48 and subsequently passes out of the transverse bore 48 into the intervertebral space 23, whereupon it hardens and forms the stabilizing portion 24 extending from the body 12.

As contemplated herein, the polymer material is biocompatible with the tissues of the patient, and has a viscosity that allows flow of the liquid polymer material through the liquid receiving bore 47 and the at least one traverse bore 48. An example of such a polymeric material that is useful in the present invention is methyl methacrylate. Curing of the liquid polymer material may occur naturally by, for example, exposing the polymer material to ambient conditions, or it may require, for instance, activation through an ultraviolet, chemical or other appropriate source.

Another aspect of the present invention is a method of inserting the vertebral spacer 10 of the present invention between adjacent vertebrae 20 of the spine to facilitate stabilizing the spine. Removal of at least a portion of an intervertebral disc 21 provides a gap for insertion of the vertebral spacer 10 therein. A portion of an adjacent vertebra 20 may also require removal to more readily accommodate the vertebral spacer 10. The slot where a portion of the vertebra and disc has been removed is defined herein as a vertebral spacer receiving slot.

The direction of insertion of the vertebral spacer 10 is selected by the surgeon according to the needs of the patient. The vertebral spacer 10 may be inserted posteriorly as shown, for example in FIG. 21, anteriorly, or laterally relative to the spinal column. The body 12 of the vertebral spacer 10 may be oriented such that during the insertion procedure the lower surface 15 and the upper surface 16 of the body 12 are normal to the adjacent vertebrae 20 and substantially out of contact with vertebral surfaces. Once inserted into a desired position in the intervertebral space 23, as shown in FIG. 22, the body 12 of the vertebral spacer 10 of the present invention may be rotated so that the lower surface 15 and the upper surface 16 of the body 12 are substantially contacting the adjacent vertebrae 20. For example, the lower surface 15 of the vertebral spacer 10 may then be in contact with the lower vertebra 20, and the upper surface 16 may support the adjacent upper vertebra 20. Optional protrusions 34 extending from the lower surface 15 and/or the upper surface 16 increase the frictional resistance between the body 12 and the adjacent vertebrae 20. The anterior face 13 of the body 12 is positioned relative to the spine to maintain a desired curvature thereof, as shown in FIGS. 21 and 22.

With respect to the above description, it is to be realized that the optimum dimensional relationships for the parts of the invention, to include variations in size, materials, shape, form, function and manner of operation, assembly, and use, are deemed readily apparent and obvious to one skilled in the art, and all equivalent relationships to those illustrated in the drawing and described in the specification are intended to be encompassed by the present invention. Further, the various components of the embodiments of the invention may be interchanged to produce further embodiments and these further embodiments are intended to be encompassed by the present invention.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A vertebral spacer comprising:
(a) a body having an upper surface and a lower surface, an anterior face and a posterior face, wherein the anterior face and the posterior face extend from the lower surface to the upper surface; and
(b) a stabilizing body extending from the body.

2. The vertebral spacer of claim 1, further comprising an attachment member connecting the body and the stabilizing body wherein the attachment member has a shaft and a head.

3. The vertebral spacer of claim 1, wherein the upper surface is non-parallel to the lower surface.

4. The vertebral spacer of claim 1, wherein the body has a rectangular cross-section.

5. The vertebral spacer of claim 1, wherein the body has an ellipsoidal cross-section.

6. The vertebral spacer of claim 1, wherein the shaft of the attachment member has an anchoring region and a rotating region.

7. The vertebral spacer of claim 2, wherein the body further comprises a first bore capable of receiving the shaft of the attachment member.

8. The vertebral spacer of claim 6, wherein the stabilizing body has a second bore capable of receiving the rotating region of the attachment member.

9. The vertebral spacer of claim 1, further comprising a locking assembly capable of interlocking the body with the stabilizing body.

10. The vertebral spacer of claim 9, wherein the locking assembly comprises a spring operated locking pin.

11. The vertebral spacer of claim 10, wherein the spring operated locking pin comprises a male member, a spring slideably disposed within a female member, and a second pin receiving bore.

12. The vertebral spacer of claim 11, wherein the spring operated locking assembly comprises a leaf spring having a locking arm, a communicating slot in the stabilizing body, and the attachment member has a receiving notch.

13. The vertebral spacer of claim 1, wherein the body has at least one protrusion capable of contacting a vertebra.

14. The vertebral spacer of claim 1, wherein the body has at least one protrusion on each of the upper surface and lower surface thereof.

15. The vertebral spacer of claim 1, wherein the body has a plurality of protrusions disposed thereon.

16. The vertebral spacer of claim 13, wherein the at least one protrusion has a cross-section selected from a triangle, a rectangle and a rounded form.

17. The vertebral spacer of claim 1, wherein the body has at least one liquid receiving bore and at least one traverse bore communicating with the at least one liquid receiving bore, and wherein the at least one traversing bore opens to the surface of the body.

18. The vertebral spacer of claim 16, wherein the stabilizing body is a hardened polymer extruded from the at least one traverse bore.

19. The vertebral spacer of claim 1, wherein the body further comprises at least one channel capable of receiving tissue growth.

20. The vertebral spacer of claim 18, wherein the channels have a tissue growth factor.

21. The vertebral spacer of claim 1, wherein the body is composed of a biocompatible material selected from the group consisting of a biocompatible polymer, a metal, bone or a combination thereof.

22. The vertebral spacer of claim 1, wherein the stabilizing body is selected from the group consisting of a biocompatible polymer, a metal, bone or a combination thereof.

23. The vertebral spacer of claim 1, wherein the stabilizing body has a recess capable of accepting the body.

24. The vertebral spacer of claim 2, wherein the attachment member is secured to the first bore of the body by an adhesive, a threaded screw, a nut, a friction joint or a pin.

25. A method of inserting a vertebral spacer according to the present invention, into a patient, comprising the steps of:
(a) inserting the body of the vertebral spacer according to claim 1 into an intervertebral space, wherein the body is substantially not contacting adjacent vertebrae; and
(b) rotating the body, thereby contacting the body with the adjacent vertebrae; and
(c) securing the body to the stabilizing body.

26. The method of claim 23, wherein the stabilizing body is rotatably connected to the body before inserting the body into an intervertebral space.

27. The method of claim 23, wherein the stabilizing body is secured to the body by delivering a liquid polymer to a liquid receiving bore in the body, and wherein the liquid polymer exudes from a traverse channel connected to the liquid receiving bore and hardens, thereby forming a stabilizing body.
